(19) United States
(12) Patent Application Publication
(10) Pub. No.: US 2025/0344836 A1
(43) Pub. Date: Nov. 13, 2025

(54) APPARATUS FOR REGULATING FLOW RATE OF A LIQUID AND A CLEANING DEVICE

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Feng Jianxin, Shenzhen (CN)

(73) Assignee: SHENZHEN NUON MEDICAL EQUIPMENT CO., LTD., Shenzhen (CN)

(21) Appl. No.: 18/822,555

(22) Filed: Sep. 3, 2024

(30) Foreign Application Priority Data
May 7, 2024 (CN) 2024105599944
May 7, 2024 (CN) 2024209719457
May 7, 2024 (CN) 2024209719777
May 7, 2024 (CN) 2024209770010

(51) Int. Cl. A46B 7/08; A46B 9/00; A61N 5/06

(52) U.S. Cl. CPC A46B 7/08; A46B 9/005; A61N 5/0613; A46B 2200/1006

(57) ABSTRACT

Embodiments of the present invention provide a cleaning apparatus for cleansing the skin. The apparatus comprises a brush assembly rotably installed in a housing. The brush assembly is driven by a driving assembly to rotate in circular manner to cleanse the skin. The brush assembly may further comprise a lifting assembly for driving the brush assembly to stretch out or retract into the housing, such that when it is necessary to use the cleaning device, the brush assembly can be controlled to extend out of the housing. During idle state, the brush assembly can be controlled to enter the housing through the lifting assembly, so as to prevent the brush assembly from being exposed, damaged or contaminated to a certain extent, so as to facilitate the storage and carrying of the cleaning device.

### TECHNICAL FIELD

The present invention relates generally to cosmetic treatment. More specifically, the present invention relates to an apparatus for cleaning skin of a user.

### BACKGROUND ART

Nowadays, there is an increasing focus on skin cleansing in personal care, and when it comes to facial cleansing, people often use facial cleansing devices to improve the effectiveness of facial cleansing. The facial cleansing device often comes with a liquid cream bottle that contains a facial cleansing product. For cleaning the skin surface prior to application of the facial cleansing product, a brush is used to foliate the skin.

The existing liquid cream bottle (such as a facial wash, cosmetic bottle, ointment etc.) comprises a bottle body and a cap body, and the liquid outlet end of the bottle body is provided with a threaded ring, and the cap body is threaded with the threaded ring to seal the outlet of the bottle body and prevent the ointment leakage after the bottle body is squeezed. In order to control the dispensing rate of the paste, an adjustment ring is set up, and the size of the outlet hole is adjusted by rotating the adjustment ring.

However, when the baffle located in the adjusting ring is subjected to excessive pressure of the paste, it is easy to cause the baffle to deform, and the paste enters the inner side of the adjusting ring, and is easy to accumulate in the gap position, breeding bacteria.

The conventional kit for cleansing the skin provides a separate brush or a brush assembly. The existing cleaning device usually uses a motor to drive the brush disc to rotate in order to clean the skin. When a user uses a cleanser for cleansing, the brush disc rotates and the bristles clean the skin. However, the bristles also make the stratum corneum of the skin thin and damaged. Therefore, it is not conducive to the skin health when there are wounds and wrinkles on the surface of the skin.

In order to facilitate the user's cleansing, some of the existing facial cleanser device comes equipped with a brush head in the bottle, and a liquid outlet hole connected with the bottle is opened in the brush head, and the skin can be cleaned through the brush head after the liquid outlet hole is squeezed out of the facial cleanser. However, the hair brush head cannot be removed from the bottle and needs to be used in conjunction with the bottle. A separate facial cleanser needs to be used in conjunction with the device. The brush is protruding, and a separate cover needs to be used to cover the outside of the brush to protect the bristles from contamination and inconvenient storage and carrying.

### OBJECTS OF THE INVENTION

Some of the objects of the invention are as follows:

An object of the present invention is to provide a, apparatus with a flow regulating mechanism to prevent the paste of the plaster liquid bottle enters the inner side of the adjusting ring.

Another object of the present invention is to provide a cleaning device that serves multiple functions.

Another object of the present invention is to provide a cleaning device with a lifting mechanism to retract and lift the brush out of a housing.

Another object of the present invention is to provide a cleaning device that provide housing for the brush when not in use.

### SUMMARY OF THE INVENTION

According to a first aspect of the present invention, an apparatus for cleaning skin of a user is provided. The apparatus comprising: a housing; a brush assembly in the housing for cleaning the skin of the user, wherein the brush assembly is rotably installed in the housing; a driving assembly to drive the brush assembly to rotate, the driving assembly is connected to the brush assembly; at least one stimulation element in the housing to provide a therapy to the user.

In one embodiment of the invention, the brush assembly comprises a first brush disc and a second brush disc, the second brush disc is rotably sleeved outside the first brush disc, and the driving assembly drives the first brush disc to rotate differentially with the second brush disc.

In one embodiment of the invention, the first brush disc comprises a support plate and a plurality of group of first bristles arranged on the support plate; the second brush disc comprises a support ring and the a plurality of group of second bristles are arranged on the support ring.

In one embodiment of the invention, the plurality of group of first bristles and the plurality of group of second bristles are transparent or translucent.

In one embodiment of the invention, the apparatus further comprising an annular seat, a first end of the annular seat is detachably connected with a liquid outlet of a bottle body and a second end of the annular seat is detachably connected with the housing.

In one embodiment of the invention, the at least one stimulation element is arranged around axis of the brush assembly.

In one embodiment of the invention, the at least one stimulation element is selected from a group consisting of Light Emitting Diodes (LEDs), lasers, heating elements, cooling elements, vibration elements, electrodes, micro-current element and combinations thereof.

According to a second aspect of the present invention, an apparatus for cleansing skin of a user is provided. The apparatus comprising: a housing; a brush assembly in the housing, the brush assembly comprising a first brush disc and a second brush disc, the second brush disc is roatably sleeved outside the first brush; a driving assembly connected with the brush assembly for driving the first brush disc to rotate differentially with the second brush disc.

In one embodiment of the invention, the first brush disc comprises a support plate and a plurality of groups of first bristles arranged on the support plate; the second brush disc comprises a support ring and the a plurality of group of second bristles are arranged on the support ring.

In one embodiment of the invention, the stiffness of the plurality of group of first bristles and the plurality of group of second bristles are different.

In one embodiment of the invention, the driving assembly further comprises: a gear assembly for driving the first brush disc to synchronously reverse rotated with the second brush disc; a driver for driving the gear assembly and a mounting pad for supporting the driver.

In one embodiment of the invention, the gear assembly comprises a driver for driving the first brush disc to rotate, an internal tooth disc for driving the second brush disc to rotate and a transmission gear that connects the internal tooth disc with the driving gear.

According to a third aspect of the present invention, an apparatus for cleaning skin of a user is provided. The apparatus comprising: a housing; a brush assembly in the housing for cleaning the skin of the user; a driving assembly connected with the brush assembly to drive the brush assembly to rotate; a lifting assembly for driving the brush assembly to retract and stretch out from the housing.

In one embodiment of the invention, the lifting assembly comprises a rotating seat and a lifting seat connected with the driving assembly, a first end of the rotating seat is rotably installed at top of the housing, and a second end of the rotating seat extends inside the housing and connects with the lifting seat.

In one embodiment of the invention, the housing comprises a guide rail for guiding the lifting seat to move vertically towards and forwards the rotating seat, when the rotating seat is rotated.

In one embodiment of the present invention, the lifting seat comprise one or more connecting arm, the one or more arm connects with the guide rail.

In one embodiment of the present invention, the lifting assembly further comprises a spring for driving the lifting seat close to the rotating seat, the spring is connected with the lifting seat and the rotating seat.

In one embodiment of the present invention, the lifting assembly further comprises one or more slide rods connected to one or more connecting blocks in the lifting seat, the one or more slide rods extend vertically upward in the housing and connects to the rotating seat.

In one embodiment of the present invention, a first end of the one or more slide rods comprises a limiting part, the one or more slide rods has a spring sleeved.

In one embodiment of the present invention, the apparatus further comprises an annular seat, a first end of the annular seat is detachably connected with a liquid outlet end of a bottle body and a second end of the annular seat is detachably connected with the housing.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other component on the piece.

In the context of the specification, the terms “first”, “second” and “third” are only used for descriptive purpose and does not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 is a schematic diagram of the three-dimensional structure of the plaster liquid bottle in accordance with an embodiment of the present invention.

FIG. 2 is a schematic diagram of the three-dimensional structure of the adjusting mechanism in accordance with an embodiment of the present invention.

FIG. 3 is an exploding view of the adjusting mechanism in FIG. 2 in accordance with an embodiment of the present invention.

FIG. 4 is the top view of the connecting seat in accordance with an embodiment of the present invention.

FIG. 5 is a schematic diagram of the three-dimensional structure of the connecting seat in accordance with an embodiment of the present invention.

FIG. 6 is a schematic diagram of the three-dimensional structure of the adjusting seat in accordance with an embodiment of the present invention.

FIG. 7 is a three-dimensional structure schematic diagram of the cleaning device, the bottle body and the adjusting mechanism in accordance with an embodiment of the present invention.

FIG. 8 is a schematic diagram of the three-dimensional structure of the cleaning device in accordance with an embodiment of the present invention.

FIG. 9 is the exploded view of the cleaning device in FIG. 8 in accordance with an embodiment of the present invention.

FIG. 10 is a schematic diagram of the three-dimensional structure of the first brush disc in accordance with an embodiment of the present invention.

FIG. 11 is a schematic diagram of the three-dimensional structure of the second brush disc in accordance with an embodiment of the present invention.

FIG. 12 is an exploded view of the drive assembly and the phototherapy assembly in accordance with an embodiment of the present invention.

FIG. 13 is an exploding view of the cleaning set according to an alternate embodiment of the present invention.

FIG. 14 is a schematic diagram of the three-dimensional structure of the shell in accordance with an embodiment of the present invention.

FIG. 15 is the three-dimensional structure schematic diagram of lifting seat and actuator in accordance with an embodiment of the present invention.

FIG. 16 is a schematic diagram of the three-dimensional structure of the bracket, the first sealing ring, the second sealing ring and the connecting piece in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose an apparatus for regulating liquid flow rate of a liquid from a bottle. The apparatus comprises a connected seat and an adjusting seat rotably connected with each other through clamping jaws engaged with strip shaped hole. One end of the connecting seat is for connecting with the bottle body and the other end is for connection with the adjusting seat. The connecting seat has a first perforation for communicating with an opening on the bottle body for dispensing liquid. The adjusting seat has a second perforation used for communicating with the first perforation. The second perforation is arranged eccentrically on the adjusting seat, such that when the adjusting seat is rotated over the connecting seat, the second perforation on the adjusting seat changes from concentric position to closed position.

Another embodiments of the present invention disclose a cleaning device for cleaning the skin of a user. The cleaning device comprises a brush assembly rotably installed in a housing. The brush assembly is driven by a driving assembly to rotate the brush assembly in a circular manner to clean the skin. The brush assembly may comprise a first brush disc having a support plate with plurality of bristles and a second brush disc having a support disc with plurality of bristles such that the second brush disc is rotably sleeved outside the first brush disc. The brush assembly further comprises a gear assembly having a driving gear for driving the first brush disc to rotate, and an internal tooth disc for driving the second brush disc to rotate. A transmission gear connects the internal tooth disc with the first brush disc, such that the internal tooth disc is coaxially connected with the second brush disc. The brush assembly further comprises an lifting assembly for driving the brush assembly to stretch out or retract into the housing. The lifting assembly is used to control the brush assembly to extend our or retract into the housing, such that when it is necessary to use the cleaning device, the brush assembly can be controlled to extend out of the housing. During idle state, the brush assembly can be controlled to enter the housing through the lifting assembly, so as to prevent the brush assembly from being exposed, damaged or contaminated to a certain extent, so as to facilitate the storage and carrying of the cleaning device. The cleaning device further comprises an annular seat, wherein one end of the annular seat is detachably connected with the liquid outlet end of the bottle body and the other end is connected with one end of the housing close to the brush assembly.

In some embodiments, a system is provided that comprises the apparatus for regulating flow rate of liquid, the cleaning device and one or more stimulation elements. The one or more stimulation elements is selected from the group consisting of Light Emitting Diodes (LEDs), lasers, heating elements, cooling elements, vibration elements, electrodes.

Several additional functionalities may also be added to the apparatus and the cleaning device. For example, the apparatus may have one or more sensors to sense the pH and monitor one or more parameters of the skin of a user. Moreover, activation and control of specific stimulation elements may be based upon feedback received from the one or more sensors, such as pressure sensors (for ascertaining contact with a body portion of the user) and temperature sensors (for maintaining the temperature of heating and/or cooling elements within permissible limits) about several parameters, such as skin temperature, treatment time, skin pH. Electrical power to operate the device may be provided through an onboard rechargeable battery that may be charged using a power cable. The apparatus can also be controlled by a user computing device communicating with the apparatus over a communication network through a communication interface built into the therapeutic device.

Several embodiments of the present invention will now be described in detail with references to FIGS.

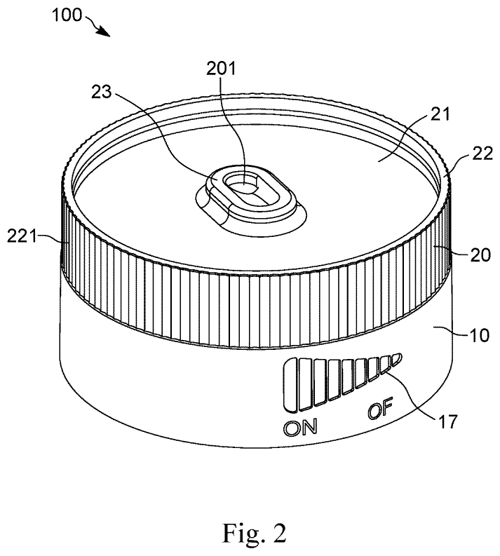

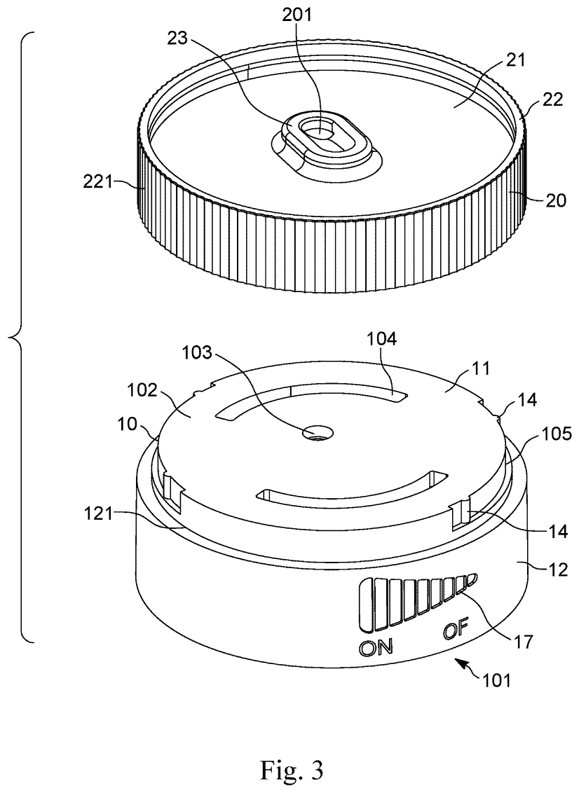

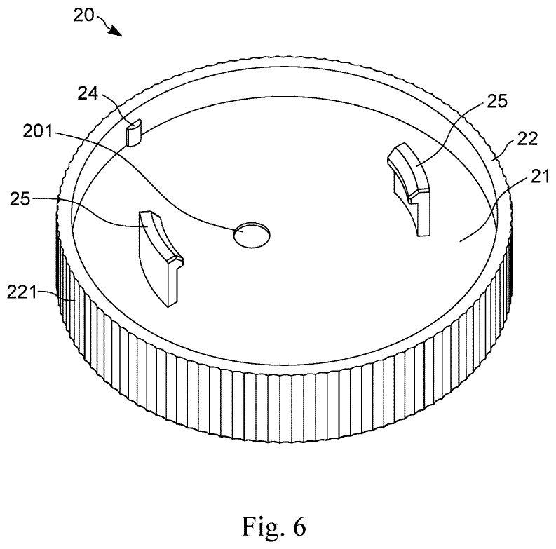

Referring to FIG. 2, FIG. 3 and FIG. 6 together, an apparatus 100 for regulating the flow rate of a liquid from a bottle provided in the embodiment of the present invention is now described. The apparatus 100 comprises a connecting seat 10 and an adjusting seat 20. The connecting seat 10 has a first end 101 and a second end 102, the first end 101 is used for connecting with the bottle body 200, and the second end 102 is arranged relative to the first end 101. The connecting seat 10 is provided with a first perforation 103, and one end of the first perforation 103 is used to communicate with the opening of the bottle body 200. The first perforation 103 is eccentrically arranged on the connecting seat 10. The adjusting seat 20 is used for adjusting the liquid flow rate of the bottle body 200. The adjusting seat 20 is rotably connected with the second end 102 of the connecting seat 10. The adjusting seat 20 is provided with a second perforation 201, the second perforation 201 is used for communicating with the other end of the first perforation 103. The second perforation 201 is eccentrically arranged on the adjusting seat 20. The adjusting seat 20 has a clamping jaw 25 that clamp in a strip-shaped hole 104 in the connecting seat 10. The strip-shaped hole 104 is round-shaped, and the circumference where the strip-shaped hole 104 is located is coaxially arranged with the connecting seat 10. The perpendicular distance between the first perforation 103 and the second perforation 201 and the axis of the connecting seat 10 is approximately equal. When the adjusting seat 20 rotates over the connecting seat 10, the second perforation 201 meet with the first perforation 103 and gradually align, so that through the relative rotation of the adjusting seat 20 and the connecting seat 10, the size of the area of the communicating part between the second perforation 201 and the first perforation 103 can be adjusted to facilitate the flow rate of the liquid paste. The connection stability between the adjusting seat 20 and the connecting seat 10 is maintained by using the clamping jaw 25 to clamp into the strip-shaped hole 104. This prevents the deformation of the position of the adjusting seat 20 near the second perforation 201 when the first perforation 103 is blocked. The gap between the connecting seat 10 and the adjusting seat 20 is avoided to prevent the liquid paste from entering the gap. The strip-shaped hole 104 is arc-shaped, and when the adjusting seat 20 is rotated relative to the connecting seat 10, the clamping jaw 25 can slide along the strip-shaped hole 104. During the rotation, the position of the second perforation 201 relative to the first perforation 103 changes from eccentric to co-centric, thus controlling the opening size for discharging the liquid.

In an embodiment of the present invention, the adjusting seat 20 comprises a plurality of clamping jaw 25 arranged in an annular array around the connecting seat 10. The connecting seat 10 comprises a plurality of strip-shaped holes; each corresponding to a clamping jaw on the adjusting seat 20. Increasing the number of clamping jaw 25 and the strip-shaped holes 104 improves the stability of connection between the adjusting seat 20 and the connecting seat 10.

In an embodiment, the clamping jaw 25 and the strip-shaped holes are two in number, located diagonally on the adjusting seat 20 and the connecting seat 10, respectively. By utilizing two clamping jaws and the strip-shaped holes, the processing of connecting seat 10 and adjusting seat 20 is simplified, and the balance of force on both sides of connecting seat 10 and adjusting seat 20 rotation axis is guaranteed.

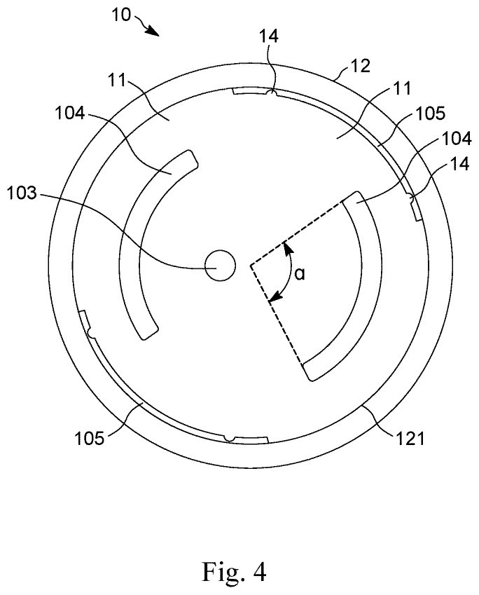

Referring to FIG. 2 to FIG. 4, the central angle α corresponding to the strip-shaped hole 104 is 60°-150°; The cross-section of the clamping jaw 25 is arc-shaped, and the ratio of the central angle corresponding to the cross-section of the clamping jaw 25 and the central angle corresponding to the strip-shaped hole 104 is 0.1-0.5. The ratio enables the clamping jaw 25 to slide along the length direction of the strip-shaped hole 104 so that the angle of rotation of the adjusting seat 20 relative to the connecting seat 10 is easily controlled, which in turn controls the distance of the second perforation 201 and the first perforation 103.

In an alternate embodiment, the central angle corresponding to the strip-shaped hole 104 is α 75°, 90°, 120° or 135°, to select a suitable central angle according to the number of strip-shaped holes 104 and the angle of rotation. The suitable central angle is needed to control the position of relative movement of the second perforation 201 and the first perforation 103.

In an alternate embodiment, the ratio of the circular center angle corresponding to the cross-section of the clamping jaw 25 and the circular center angle corresponding to the strip-shaped hole 104 is 0.2, 0.3 or 0.4, so that the stability of the clamping jaw 25 along the axis direction of the adjusting seat 20 in the strip-shaped hole 104 can be maintained; the stability of the connection between the adjusting seat 20 and the connecting seat 10 is improved; and the rotation angle of the adjusting seat 20 is guaranteed.

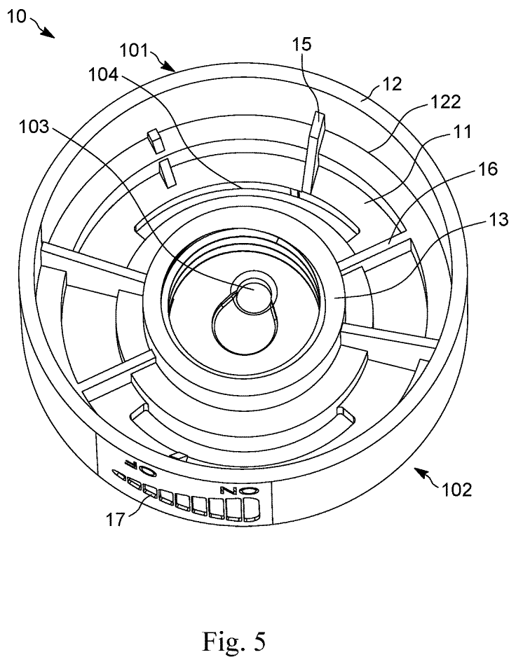

Referring to FIG. 2, FIG. 3 and FIG. 5, the connecting seat 10 comprises a first supporting plate 11 and a first annular wall 12 arranged along the edge of the first supporting plate 11, and the first perforation 103 and a strip-shaped hole 104 are arranged on the first supporting plate 11. By adopting the first annular wall 12, the thickness of the first supporting plate 11 can be reduced, and the structural strength of the connecting seat 10 is enhanced. When the first annular wall 12 is matched with the bottle body 200, the stability of the connection seat 10 and the bottle body 200 can be improved.

Referring to FIG. 2, FIG. 3 and FIG. 6, the adjusting seat 20 comprises a second supporting plate 21 coaxially arranged with the first supporting plate 11 and a second annular wall 22 arranged along the edge of the second supporting plate 21. The second perforation 201 is formed on the second supporting plate 21, and the clamping jaw 25 is arranged on one side of the second supporting plate 21 close to the first supporting plate 11. The structural strength of the edge of the second supporting plate 21 can be enhanced by adopting the second annular wall 22, and the stability of the connection between the adjusting seat 20 and the connecting seat 10 is conducive to being enhanced. Optionally, the outer diameter of the second annular wall 22 is equal to the outer diameter of the first annular wall 12, so that the size of the adjusting seat 20 can be matched with the size of the connecting seat 10.

Referring to FIG. 2, FIG. 3 and FIG. 6, the first annular wall 12 is located on one side of the first supporting plate 11 away from the adjusting seat 20, and the second supporting plate 21 is located at the middle part of the second annular wall 22 along the length direction. One end of the first annular wall 12 close to the adjusting seat 20 is provided with a first step structure 121, and one end of the second annular wall 22 close to the first supporting plate 11 is sleeved on the first step structure 121. The second supporting plate 21 is located in the middle of the second annular wall 22 along the length direction, and is not limited to the second supporting plate 21 being located in the middle position of the second annular wall 22 along the length direction. The length direction of the second annular wall 22 is consistent with the axis direction of the second annular wall 22. By adopting the second annular wall 22 to be arranged on the first step structure 121, the stability of the rotation of the connecting seat 10 and the adjusting seat 20 is improved.

Referring to FIG. 2, FIG. 3 and FIG. 6, the connecting seat 10 further comprises a first reinforcing rib 15 connecting the first supporting plate 11 and the first annular wall 12. Optionally, the first reinforcing ribs 15 are plurality in number, and the plurality of first reinforcing ribs 15 are arranged around the axis of the first supporting plate 11. Adopting the first reinforcing rib 15 is conducive to improving the stability of the connection between the first annular wall 12 and the first supporting plate 11.

Optionally, one side of the first supporting plate 11, far away from the second supporting plate 21, is provided with a connecting ring 13 for communicating with the opening of the bottle body 200. The first perforation 103 is located in the connecting ring 13. The connecting seat 10 further comprises a reinforcing plate 16, the reinforcing plate 16 is dislocated with the strip-shaped hole 104, the two ends of the reinforcing plate 16 are connected with the first annular wall 12 and the connecting ring 13, respectively. One side of the reinforcing plate 16 is connected with the first supporting plate 11. The dislocation arrangement of the reinforcing plate 16 and the strip-shaped hole 104 refers to the position of the reinforcing plate 16 on the first supporting plate 11 without opening the strip-shaped hole 104. When the strip-shaped holes 104 are plurality in number, the reinforcing plate 16 is positioned between two adjacent strip-shaped holes 104. In this way, the stability of the first supporting plate 11 can be enhanced; the connecting seat 10 is prevented from deformation; and the reinforcing plate 16 is avoided from interfering with the movement of the clamping jaw 25. Optionally, the connecting ring 13 can be connected with the bottle body 200 by means of a threaded fit, clamping fit, or welding to keep the connection stable between the apparatus 100 and the bottle body 200.

The connecting seat 10 further comprises a second reinforcing rib connecting the first supporting plate 11 and the connecting ring 13 to enhance the stability of the connecting ring 13 and the first support plate 11. The connecting seat 10 comprises a plurality of the second reinforcing ribs arranged around the connecting ring 13 so that the stability of the connecting ring 13 and the first supporting plate 11 connection is conducive, and the stress balance is maintained.

Referring to FIG. 2, FIG. 3 and FIG. 6, the adjusting seat 20 is provided with a second protrusion 24, and the connecting seat 10 is provided with a first protrusion 14 that coordinates the position of the adjusting seat 20 with the second protrusion 24. The first protrusion 14 and the second protrusion 24 may be convex bars. The first protrusion 14 is arranged along the length direction of the first annular wall 12, and the second protrusion 24 is arranged along the linear direction of the second annular wall 22. The first protrusion 14 can be matched with the second protrusion 24 to locate the adjusting seat 20 rotation position to locate the opening or closing position of the second perforation 201 and the first perforation 103. By adopting the second protrusion 24 and the first protrusion 14 to cooperate, the position of the adjusting seat 20 rotation can be located.

Referring to FIG. 2, FIG. 3 and FIG. 6, the connecting seat 10 is provided with an avoidance groove 105 for avoiding the second protrusion 24. The first protrusion 14 is arranged around the avoidance groove 105. By setting up the avoidance groove 105, the second protrusion 24 can be avoided, and the wear of the second protrusion 24 is reduced. Specifically, the first protrusion 14 and the avoidance groove 105 are located at one end of the first annular wall 12 close to the second supporting plate 21. The avoidance groove 105 is arc-shaped, and the central angle corresponding to the avoidance groove 105 is greater than or equal to the central angle corresponding to the strip-shaped hole 104, so that the angle range of rotation of the adjusting seat 20 can be conveniently controlled through the strip-shaped hole 104.

Referring to FIG. 2, FIG. 3 and FIG. 6, the first protrusion 14 are two in number positioned at two opposing ends of the avoidance groove 105. The first protrusion 14 positioned at a first end of the avoidance groove 105 indicates a position when the second perforation 201 is disconnected from the first perforation 103. The first protrusion 14 positioned at a second end of the avoidance groove 105 indicates a position when the second perforation 201 is co-centric with the first perforation 103. This facilitates the convenient positioning of the start and stop position of opening or closing the second perforation 201 of adjusting seat 20.

In some embodiment, the avoidance groove 105 may also be arranged on the second annular wall 22, and two second protrusions 24 are arranged in the avoidance groove so as to facilitate the positioning of the rotation position of the screwing adjustment seat 20.

In an embodiment of the present application, the adjusting seat 20 comprises two second protrusions 24 relatively positioned at the two opposing sides of the adjusting seat 20 along the radial direction. The avoidance groove 105 are two in number, each corresponds to the second protrusion 24. The first protrusion 14 is arranged in each of the avoidance groove 105.

Referring to FIG. 2, FIG. 3 and FIG. 6, the periphery of the first annular wall 12 is provided with an indicator 17 for indicating the rotating position of the adjusting seat 20. In this way, it is convenient to adjust the rotation position of the adjusting seat 20, and the size of the communicating part of the second perforation 201 and the first perforation 103 is controlled. Optionally, a second step structure 122 is arranged in the first annular wall 12, and the second step structure 122 is located on one side of the first support plate 11, which is far away from the second support plate 21. In this way, the structural strength of the first annular wall 12 and the first supporting plate 11 can be increased.

Referring to FIG. 2, FIG. 3 and FIG. 6, the periphery of the second annular wall 22 is provided with anti-skid 221 that plays an anti-slip effect, so that the user can rotate the adjusting seat 20.

Referring to FIG. 2, FIG. 3 and FIG. 6, the middle part of the adjusting seat 20 is provided with a boss 23, and the second perforation 201 penetrates through the boss 23. Specifically, the boss 23 is convex on one side of the second support plate 21 far away from the first support plate 11, so that the structural strength of the second support plate 21 near the position of the second perforation 201 is made to prevent the second support plate 21 from deforming near the second perforation 201.

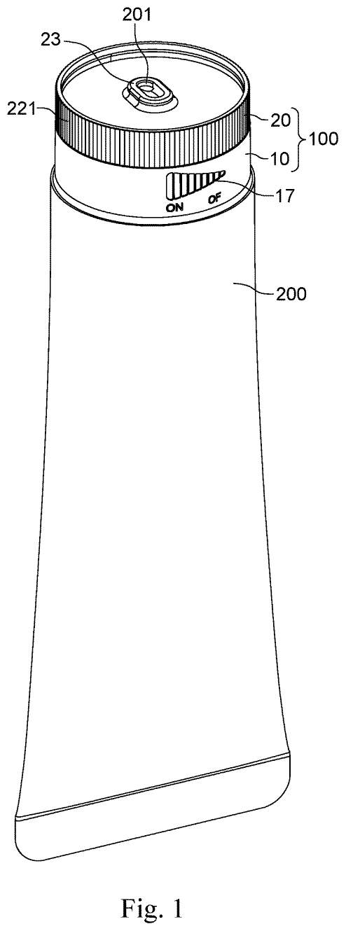

Referring to FIG. 1 to FIG. 3, the embodiment of the present invention also provides a liquid bottle, comprising a bottle body 200 with the apparatus 100 for regulating the flow rate of the liquid described above embodiments. The bottle body 200 is connected with the first end 101 of the connecting seat 10, and the opening of the bottle body 200 is in communication with the first perforation 103. The liquid flow rate of bottle body 200 can be controlled by apparatus 100 to meet the usage habits of different users. The opening position of the bottle body 200 can be provided with a threaded ring, and the connecting seat 10 threads are matched by the threaded ring to be connected with the bottle body 200.

Referring to FIG. 1 to FIG. 3, the bottle body 200 further comprises a lid body, and the lid body cover is at one end of the second annular wall 22, away from the connecting seat 10. The second perforation 201 can be easily covered to prevent paste contamination. The outer diameter of the cap body is equal to the outer diameter of the second annular wall 22, so it is convenient to prevent the inverted plaster bottle.

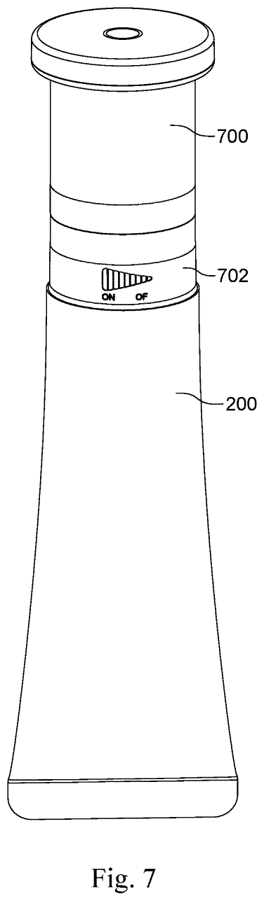

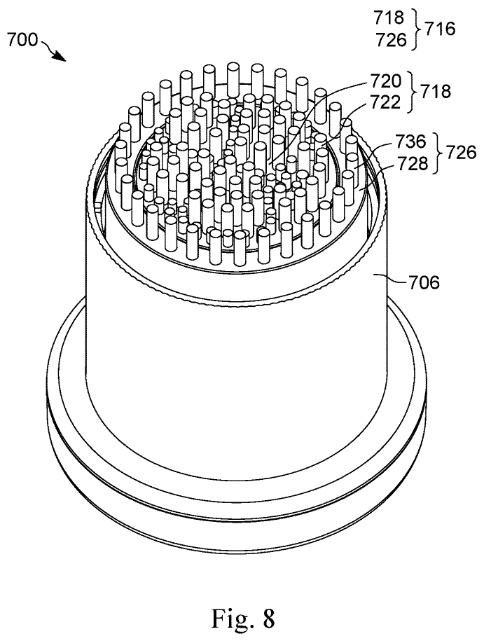

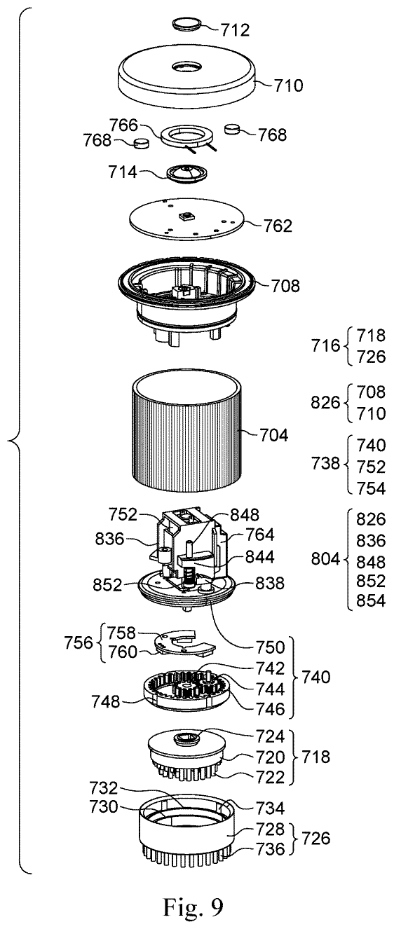

Referring to FIG. 7, FIG. 8 and FIG. 9, the cleaning device 700 provided in the embodiment of the present invention is described. The cleaning device 700 comprises a housing 704, a brush assembly 716, a driving assembly 738 and one or more stimulation elements 756. The brush assembly 716 is rotably installed in the housing 704. The driving assembly 738 is used for driving the brush assembly 716 to rotate. The driving assembly 738 is installed in the housing 704, and the power output end of the driving assembly 738 is connected with the brush assembly 716. The one or more stimulation elements 756 is used for performing alternate therapy on the skin. The one or more stimulation elements 756 is installed in the housing 704. The driving assembly 738 is used to drive the brush assembly 716, to clean the skin in a circle, so as to realize the skin cleansing. The one or more stimulation elements 756 are used to perform alternate therapy on the skin to facilitate the treatment of the skin, to increase the function of the cleaning device 700, to help reduce the impact on wounds and wrinkles during the cleaning process, and to improve the user experience.

Referring to FIG. 8 to FIG. 11, the brush assembly 716 comprises a first brush disc 718 and a second brush disc 726 arranged around the first brush disc 718. The second brush disc 726 is rotably sleeved outside the first brush disc 718. The first brush disc 718 is connected with the power output end of the second brush disc 726 and the driving assembly 738. The driving assembly 738 is used for driving the first brush disc 718 and the second brush disc 726 to rotate at differential speed. The first brush disc 718 and the second brush disc 726 may rotate in the same direction with different angular velocities, or rotate with the same or different angular velocities in different directions. The second brush disc 726 is rotably sleeved outside the first brush disc 718, so that the first brush disc 718 and the second brush disc 726 can rotate relatively, that is, can rotate at differential speed. The first brush disc 718 and the second brush disc 726 are driven to rotate at differential speed through the driving assembly 738, so that the first brush disc 718 and the second brush disc 726 rotate at different speeds, in order to provide a better cleaning effect in the process of cleaning the skin.

Referring to FIG. 8 to FIG. 11, the driving assembly 738 comprises a gear assembly 740, a driver 752 and a mounting pad 754. The gear assembly 740 is used for driving the first brush disc 718 to synchronously reverse rotate with the second brush disc 726. The driver 752 is used for driving the gear assembly 740, the mounting pad 754 supports the driver 752. The mounting pad 754 is connected with the housing 704, and the power take-off end of the driver 752 is connected with the gear assembly 740. The first brush disc 718 and the second brush disc 726 rotate synchronously in reverse direction, and the rotation angular velocity of the first brush disc 718 and the second brush disc 726 is not limited. Since the first brush disc 718 and the second brush disc 726 rotate in opposite directions, the skin can be brushed from different directions so that the cleaning effect can be improved.

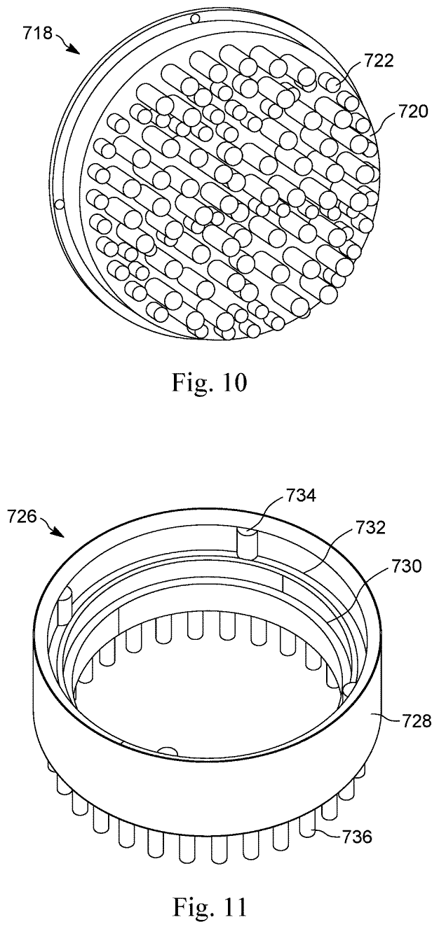

Referring to FIG. 8, FIG. 10 and FIG. 11, the first brush disc 718 comprises a support plate 720 and a plurality of groups of first bristles 722 arranged on the support plate 720; the second brush disc 726 comprises a support ring 728 and a plurality of groups of second bristles 736 arranged on the support ring 728. The support ring 728 is arranged on the support plate 720, and the first step structure 730 of the positioning support plate 720 is arranged in the support ring 728. The support plate 720 helps fix the plurality of groups of first bristles 722 conveniently, to facilitate the synchronous rotation of a plurality of groups of first bristles 722. The support ring 728 helps fix the plurality of groups of second bristles 736 conveniently, to control the synchronous rotation of a plurality of groups of second bristles 736. Using the first step structure 730, it is convenient to locate the position of the support plate 720 in the support ring 728 to control the height position of the first bristles 722 and the second bristles 736.

Alternatively, a plurality of first bristle arrays are arranged on a support plate 720, and the height of a part of the first bristles 722 in the plurality of groups is higher than the height of the other part of the first bristles 722. For example, the shorter part of the first bristle 722 can divide the higher first bristle 722 into multiple areas, such as forming a four-leaf clover shape, a fan blade shape, etc. In this way, during the first brush disc 718 rotation, the pressure of the first bristle 722 on the skin can change periodically, which can help improve the cleaning effect.

In an embodiment of the present invention, the plurality of first bristles are harder and stiffer than the plurality of second bristles. Alternatively, the plurality of second bristles can be harder than the plurality of first bristles.

Alternatively, the support ring 728 is rotably inserted at the open end of the housing 704. In this way, the stability of the rotation of the support ring 728 relative to the housing 704 can be improved, the swing of the second brush disc 726 can be reduced, and then the stability of the operation of the first brush disc 718 and the second brush disc 726 can be improved, and noise and wear are reduced.

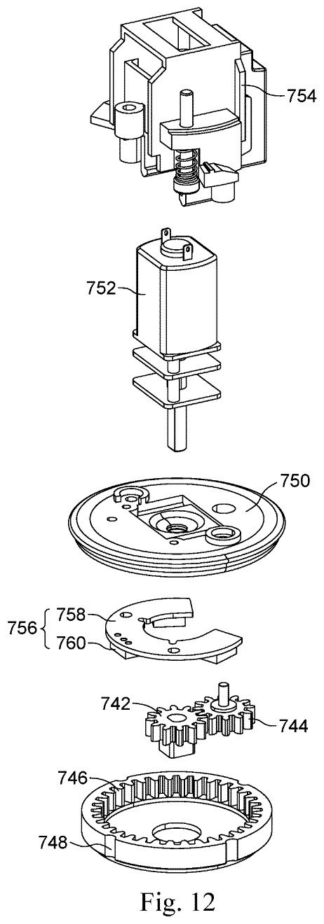

Referring to FIG. 8, FIG. 9 and FIG. 12, the gear assembly 740 comprises a driving gear 742 for driving the first brush disc 718 to rotate, an internal toothed disc 746 for driving the second brush disc 726 to rotate, a transmission gear 744 for the transmission connection with the internal toothed disc 746 and a driving gear 742. An output shaft of the driver 752 of the driving gear 742 is connected with the first brush disc 718, and the internal toothed disc 746 is coaxially connected with the second brush disc 726. Using the driver 752, the synchronous reverse rotation of the first brush disc 718 and the second brush disc 726 can be realized.

Referring to FIG. 8, FIG. 9 and FIG. 12, the second brush disc 726 is sleeved on the internal toothed disc 746. The second brush disc 726 is provided with a positioning protrusion 734, and the internal toothed disc 746 is provided with a positioning groove 748 for the positioning protrusion 734 to be clamped in. By adopting a positioning protrusion 734 to cooperate with clamping into the positioning groove 748, when the internal toothed disc 746 rotates, the second brush disc 726 can be driven to rotate synchronously. In an alternate embodiment, the one end of the internal toothed disc 746 close to the first brush disc 718 may be sleeved outside the second brush disc 726, a positioning protrusion may be arranged on the internal toothed disc 746, and a positioning groove may be arranged on the second brush disc 726, so that the second brush disc 726 can be driven to rotate through the internal toothed disc 746.

Specifically, the support ring 728 is arranged on the internal toothed disc 746. The support ring 728 is provided with a second step structure 732, and the second step structure 732 cooperates with positioning the internal toothed disc 746. In this way, the position of the second brush disc 726 along the axial direction of the internal toothed disc 746 can be accurately located.

Optionally, the positioning protrusion 734 is a convex bar arranged along the axis direction of the internal toothed disc 746, and the positioning groove 748 is strip-shaped. This is conducive to improving the stability of the connection between the internal toothed disc 746 and the support ring 728, and it reduces the wear of the positioning protrusion 734.

Referring to FIG. 8, FIG. 9 and FIG. 12, the second brush disc 726 is sleeved on the internal toothed disc 746. The internal toothed disc 746 is provided with a positioning protrusion, and the second brush disc 726 is provided with a positioning groove for the positioning protrusion to be clamped in. By adopting positioning protrusion to fit into the positioning groove, when the internal toothed disc 746 rotates, the second brush disc 726 can be driven to rotate synchronously. In alternate embodiment, the one end of the internal toothed disc 746 close to the first brush disc 718 may be sleeved outside the second brush disc 726. A positioning protrusion may be arranged on the second brush disc 726, and a positioning groove may be arranged on the internal toothed disc 746 so that the second brush disc 726 can be driven to rotate through the internal toothed disc 746.

Referring to FIG. 8, FIG. 9 and FIG. 12, the gear assembly 740 is provided with a cover plate 750 connected with the mounting pad 754, and the one or more stimulation elements 756 is connected with the cover plate 750. In this way, the stability of the connection between the one or more stimulation element 756 and the mounting pad 754 is maintained, and the one or more stimulation element 756 is prevented from rotating when the driving assembly 738 is running, so that the connection of the line of the one or more stimulation element 756 is convenient.

Referring to FIG. 8, FIG. 9 and FIG. 12, a limiting ring 724 is arranged on the support plate 720, and the internal toothed disc 746 is rotably sleeved outside the limiting ring 724. Using this arrangement, the position of the limiting ring 724 and the internal toothed disc 746 can be kept stable along the axis direction of the internal toothed disc 746. Optionally, special-shaped holes (such as rectangular holes, triangular holes or semicircular holes) are formed in the limiting ring 724, and the special-shaped holes are matched with the output shaft of the driver 752 to drive the first brush disc 718 to rotate synchronously when the output shaft of the driver 752 rotates.

In an exemplary embodiment, the one or more stimulation element 756 is a phototherapy assembly. The phototherapy assembly comprises a lamp board 758 and a plurality of LED lamp beads 760 arranged on the lamp board 758, a plurality of LED lamp beads 760 is arranged around the axis of the brush assembly 716, and the brush assembly 716 forms a light-transmitting area corresponding to the position of the LED lamp beads 760. The support plate 720 forms a light-transmitting area, and the support plate 720 can be a transparent plate, so as to facilitate the emission of phototherapy light. Optionally, LED lamp bead 760 is red LED lamp bead that can play a wrinkle removal effect, so that wrinkle removal phototherapy is carried out at the same time as facial cleansing, which is conducive to improving the wrinkle removal effect. The blue LED lamp beads 760 can used to treat the skin by using the anti-inflammatory and acne effect of blue light.

In another embodiment, the internal toothed disc 746 comprises an internal toothed ring and a sealing disc. The cover plate 750 and the sealing disc are respectively covered at two ends of the internal toothed ring to realize the sealing of the internal toothed ring. The sealing disc can be a transparent plate, so that the output of phototherapy light is convenient. The first bristles 722 and the second bristles 736 can be transparent or translucent, which facilitates the conduction of light to the skin.

Referring to FIG. 9, the cleaning device 700 further comprises a circuit board 762 and a battery 764 that is electrically connected with the circuit board 762. The circuit board 762 and the battery 764 are installed in the housing 704. The circuit board 762 is electrically connected with the battery 764, and the driving assembly 738 is electrically connected with the one or more stimulation elements 756. The battery 764 is used to facilitate the use of the cleaning device 700 in the absence of an external power supply.

Referring to FIG. 9, the cleaning device 700 further comprises a wireless charging coil 766 installed in the housing 704, and the wireless charging coil 766 is electrically connected with the circuit board 762. The wireless charging coil 766 eliminates the need for additional opening and sealing to the housing 704, which is conducive to improving the waterproof performance of the cleaning device 700.

In an embodiment of the present invention, the wireless charging coil 766 and the brush assembly 716 are respectively positioned at two ends of the housing 704 along the length direction, which facilitates the cleaning mechanism to be placed on the wireless charging dock for charging.

Referring to FIG. 9-12, a magnetic suction piece 768 is installed in the housing 704, and the magnetic suction piece 768 is used for magnetic coupling with the magnetic part on the wireless charging base. The position of the cleaning device 700 and the wireless charging dock can be kept stable, so that the wireless charging dock can wirelessly charge the cleaning device 700. Optionally, the device comprises a plurality of magnetic suction pieces 768, forming an annular array around the wireless charging coil 766.

Referring to FIG. 8, FIG. 9 and FIG. 12, the housing 704 comprises a shell 706, a bracket 708 and a buckle cover 710. The brush assembly 716 is installed at one end of the accommodating shell 706. The driving assembly 738 is installed in the accommodating shell 706, one end of the bracket 708 is inserted into the other end of the accommodating shell 706, and the buckle cover 710 is covered at the other end of the bracket 708. The circuit board 762 and the wireless charging coil 766 are mounted on the bracket 708.

Alternatively, the circuit board 762 is provided with a switching member, a pressing cap 712 is arranged on the buckle cover 710, an elastic part 714 is arranged in the buckle cover 710, and the elastic part 714 is positioned between the pressing cap 712 and the switching member. The elastic part 714 can be a silicone cap or a rubber cap, so that the inside side of the buckle cover 710 can be pasted through its end to ensure the sealing of the position of the buckle cover 710 corresponding to the pressing cap 712.

Referring to FIG. 7 to FIG. 9, the cleaning device 700 further comprises a bottle body 200 for accommodating a facial cleanser and the apparatus 702 for regulating the flow rate of facial cleanser. The apparatus 702 is installed at the liquid outlet end of the bottle body 200, and the housing 704 is connected with the apparatus 702. In this way, a complete set of cleansing products can be formed, which is convenient for users to store and carry. The rate of dispensing can be controlled by the apparatus 702, and the brush assembly 716 is prevented from contaminating the cleanser.

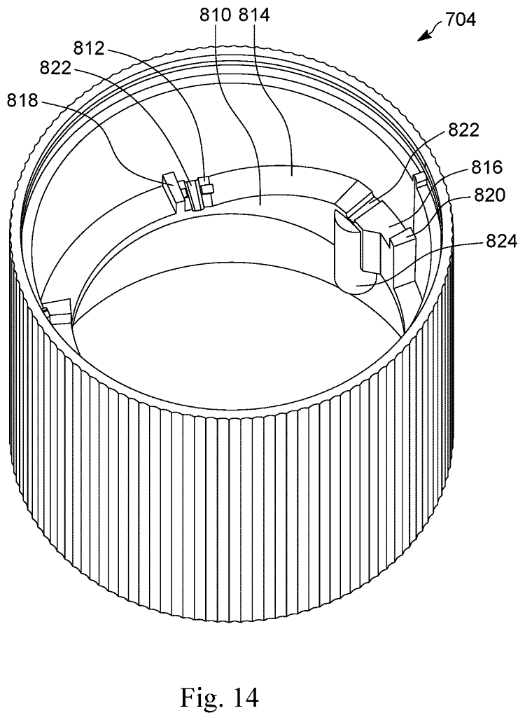

Referring to FIG. 8, FIG. 9 and FIG. 14, the cleaning device with adjusting mechanism is provided. The cleaning device 700 comprises an annular seat 802 and a bottle body 200, a housing 704, a brush assembly 716, a driving assembly 738 and an lifting assembly 804. The driving assembly 738 is positioned in the housing and is used for driving the brush assembly 716. The power output end of the driving assembly 738 is connected with the brush assembly 716. The lifting assembly 804 is used for driving the brush assembly 716 to stretch out or enter the housing 704. The lifting assembly 804 is installed in the housing 704, and the lifting assembly 804 is connected with the driving assembly 738. The bottle body 200 is used for storing the paste (such as facial cleanser, etc.), and one end of the annular seat 802 is detachably connected with the liquid outlet end of the bottle body 200, and the other end of the bottle body 200 is detachably connected with one end of the housing 704 close to the brush assembly 716. The lifting assembly 804 is used to control the brush assembly 716 to stretch out or be included in the housing 704. When the cleaning device 700 needs to be used, the brush assembly 716 can be controlled to stretch out the housing 704, so as to facilitate the cleaning of the skin; During storage, the brush assembly 716 can be retracted to the housing 704 by the lifting assembly 804, so as to prevent the brush assembly 716 from being exposed, damaged or contaminated to a certain extent, so that the storage and carrying of the cleaning set are convenient. The annular seat 802 is detachably connected with the bottle body 200 and the housing 704, so that the distance between the liquid outlet of bottle body 200 and brush assembly 716 is maintained during storage. After disassembling the annular seat 802, the liquid outlet of brush assembly 716 and bottle body 200 is exposed, so that the paste liquid is conveniently applied on brush assembly 716, and brush assembly 716 is exposed to facilitate skin cleaning.

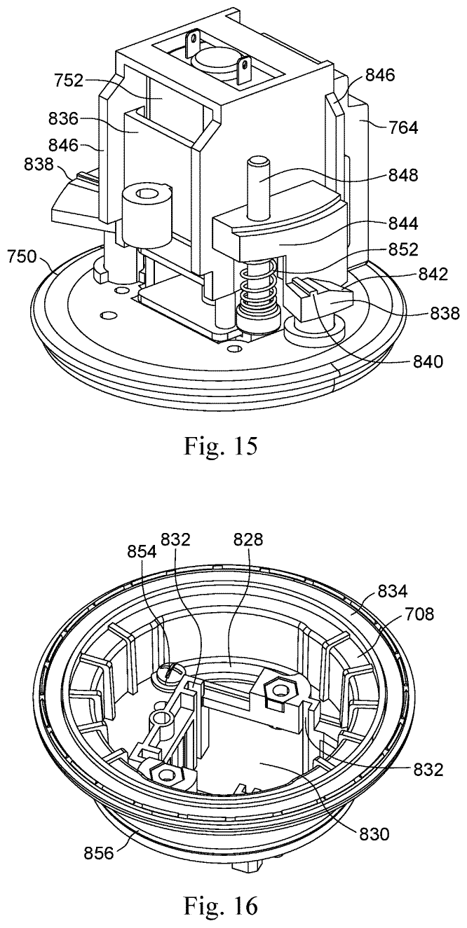

Referring to FIG. 9, FIG. 14 and FIG. 15, the lifting assembly 804 comprises a rotating seat 826 and a lifting seat 836 connected with the driving assembly 738. One end of the rotating seat 826 is rotably inserted at one end of the housing 704 away from the brush assembly 716, the other end of the rotating seat 826 extends out of the housing 704. The one end of the rotating seat 826 extending into the housing 704 is slidably connected with the lifting seat 836. The housing 704 is provided with a guide rail 810 to guide the lifting seat 836 to approach or stay away from the rotating seat 826 when the rotating seat 826 rotates. The lifting seat 836 is in a sliding fit with the guide rail 810. When the rotating seat 826 rotates, the lifting seat 836 rotates with the rotating seat 826, and the lifting seat 836 can slide along the guide rail 810 in the outward direction or inward direction, thereby pushing out the brush assembly 716 or retracting the brush assembly 716 in the housing depending on the movement of the brush assembly 716.

Referring to FIG. 9, FIG. 14 and FIG. 15, the lifting seat 836 is provided with a connecting arm 838 that is sliding-matched with the guide rail 810. The connecting arm 838 is located on one side of the guide rail 810 close to the brush assembly 716, and the connecting arm 838 is slidable on the side of the guide rail 810 that is at distant end from the rotating seat 826. The lifting assembly 804 further comprises a spring 852 for driving the lifting seat 836 close to the rotating seat 826. The spring 852 is connected with the lifting seat 836 and the rotating seat 826. When the rotating seat 826 rotates in the forward direction, the lifting seat 836 is guided to move towards the brush assembly 716. The spring 852 drives the connecting arm 838 to connect with the guide rail 810, so that the lifting seat 836 can be easily controlled to reset when the rotating seat 826 rotates in the opposite direction, and the processing of the housing 704 and the assembly of the lifting seat 836 can be convenient.

In an embodiment, the cleaning device comprise two guide rails 810 positioned at two opposing side of the lifting seat 836 and each of the guide rails 810 has corresponding connecting arm 838.

Referring to FIG. 9, FIG. 14 and FIG. 15, the lifting assembly 804 further comprises a slide rod 848 connected with the rotating seat 826. The slide rod 848 is arranged along the linear direction of the housing 704, and extends into the housing 704. The lifting seat 836 is provided with a connecting block 844 that is slidably connected with the slide rod 848. The lifting seat 836 can be guided to slide in a straight line on the rotating seat 826 through the slide rod 848 to keep the lifting seat 836 stable during movement.

In an embodiment, the cleaning device has two slide rods 848 positioned diagonally on the lifting seat 836. Each of the slide rods 848 has a corresponding connecting block 844 to improve the stability when the lifting seat 836 and the rotating seat 826 slide relatively.

In an embodiment, the connecting arm 838 is connected with the connecting block 844 to improve the structural strength of the connecting arm 838.

In an embodiment, a limiting portion 850 is arranged at one end of the slide rod 848 close to the brush assembly 716. The spring 852 is sleeved on the slide rod 848, the connecting block 844 is located at one end of the spring 852 close to the rotating seat 826. The two ends of the spring 852 are respectively countered with the limiting portion 850 and the connecting block 844 to control the compression or elongation of the spring 852 along the slide rod 848, and provide stability to the spring 852 position. In an embodiment, the slide rod 848 can be a screw.

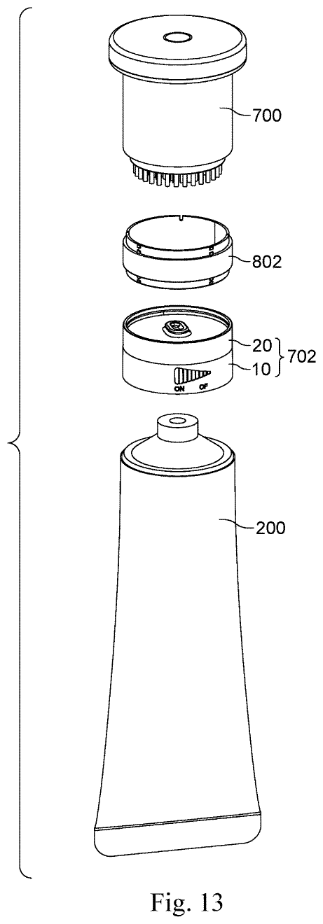

Referring to FIG. 7, FIG. 8 and FIG. 13, one side of the connecting arm 838 close to the rotating seat 826 is provided with a first clamping 840. The two ends of the guide rail 810 has the first clamping groove 822 for clamping the first clamping 840. When the connecting arm 838 rotates to the two ends of the guide rail 810, the stabilizing position of the connecting arm 838 at the end of the guide rail 810 is maintained that stabilizes the position of the brush assembly 716 when it is stretched out and retracted into the housing 704.

In an embodiment, the connecting arm 838 is provided with an inclined plane 842 matched with the guide rail 810, and the contact area between the connecting arm 838 and the guide rail 810 is increased when sliding relative to each other through the inclined plane 842, so that the stability in the sliding process is improved, and the wear and noise in the sliding process are reduced.

Referring to FIG. 9, FIG. 14 and FIG. 15, the guide rail 810 comprises a spiral section 814, a first positioning segment 812 located at one end of the spiral section 814 and a second positioning segment 816 arranged at the other end of the spiral section 814. The angle between one side of the first positioning segment 812 close to the connecting arm 838 and the radial surface of the brush assembly 716 is less than the angle between one side of the spiral section 814 close to the connecting arm 838 and the radial surface of the brush assembly 716. The angle between one side of the second positioning segment 816 close to the connecting arm 838 and the radial plane of the brush assembly 716 is smaller than the angle between one side of the spiral section 814 close to the connecting arm 838 and the radial surface of the brush assembly 716. One side of the first positioning segment 812 and the second positioning segment 816 and the connecting arm 838 sliding fit is relatively flat, and the one side of the spiral section 814 and the connecting arm 838 sliding fit is inclined to a larger amplitude. Through the spiral section 814, the ratio of the rotation angle of the rotating seat 826 and the moving distance of the lifting seat 836 along the axis direction of the housing 704 can be controlled, and through the first positioning segment 812 and the second positioning segment 816, the connecting arm 838 can be more stable when rotating to the first positioning segment 812 and the second positioning segment 816, so that the brush assembly 716 can remain stable when stretching out and entering the housing 704.

In an embodiment, one side of the first positioning segment 812 and the second positioning segment 816 sliding fit with the connecting arm 838 may be perpendicular to the axis of the brush assembly 716 to prevent the connecting arm 838 from sliding at both ends of the guide rail 810.

In an embodiment, one side of the first positioning segment 812 and the second positioning segment 816 close to the brush assembly 716 is provided with a first clamping groove 822, so, when the lifting assembly 804 rotates to the brush assembly 716 to stretch out or enter the housing 704, the connecting arm 838 is at the first positioning segment 812 or the second positioning segment 816, and the first clamping 840 is clamped into the corresponding first clamping groove 822 in coordination, to improve the stability of the connecting arm 838 at the end position of the guide rail 810.

Referring to FIG. 9, FIG. 14 and FIG. 15, one end of the guide rail 810 is provided with a first gear convex 818 for stop position of the connecting arm 838, and the other end of the guide rail 810 is provided with a second gear convex 820 for the stop position of the connecting arm 838. The first gear convex 818 and the second gear convex 820 are used to prevent the connecting arm 838 from separating from the guide rail 810. Specifically, the first gear convex 818 is located at one end of the first positioning segment 812 that is distant from the spiral section 814, and close to the brush assembly 716. The second gear convex 820 is positioned at one end of the second positioning segment 816 that is distant, from the spiral section 814 and close to the brush assembly 716.

Referring to FIG. 14, FIG. 15 and FIG. 16, the rotating seat 826 has a strip-shaped hole 828. The strip-shaped hole 828 is round-arc-shaped, and a connecting piece 854 connected with the housing 704 is sliding-inserted in the strip-shaped hole 828. The connecting piece 854 can slide in the strip-shaped hole 828 to guide the rotating seat 826 to rotate in the direction of the housing 704, to control the rotation angle range, and to improve the stability between the rotating seat 826 and the housing 704. In an embodiment, a connecting column 824 is positioned on one side of the guide rail 810 that is at distant end from the brush assembly 716, and a connecting piece 854 is connected with the connecting column 824. One end of the connecting column 824, at distant from the guide rail 810, passes through the strip-shaped hole 828, and a limiting cap is arranged, to prevent the lifting seat 836 from being separated from the housing 704. The connecting piece 854 can be a screw, and the connecting column 824 is a threaded column, to conveniently assemble or disassemble the lifting seat 836 and the housing 704.

In an embodiment the rotating seat 826 has two strip-shaped holes 828 positioned diagonally to improve the stability of the relative rotation of the rotating seat 826 and the housing 704.

In an embodiment, a guide bar 846 is arranged on the lifting seat 836 in linear direction of the housing 704. The rotating seat 826 is provided with a guide groove 832, and the guide bar 846 is slidably inserted into the guide groove 832 to improve the stability of the connection between the lifting seat 836 and the rotating seat 826, to make the lifting seat 836 more stable when sliding, and to prevent the lifting seat 836 from rotating relative to the rotating seat 826.

In an embodiment, a slide hole 830 is formed in the rotating seat 826, and the lifting seat 836 is slidably inserted in the slide hole 830 to improve the stability between the rotating seat 826 and the lifting seat 836. The guide bar 846 are plurality in number and arranged around the driving assembly 738. Each of the plurality of guide bars 846 has a corresponding guide grooves 832 to improve the stability of lifting seat 836 while lifting.

Referring to FIG. 9 and FIG. 16, the rotating seat 826 comprises a bracket 708 and a buckle cover 710, one end of the bracket 708 is inserted into one end of the housing 704 distant from the brush assembly 716. The buckle cover 710 is covered at the other end of the bracket 708, and the bracket 708 is slidably connected with the lifting seat 836. The circuit board 762 and the wireless charging coil 766 are mounted on the bracket 708.

In an embodiment, the bracket 708 is sleeved with a first sealing ring 856. The gap between the first sealing ring 856 and the inner wall of the housing 704 is filled with the bracket 708. A second sealing ring 834 is installed between the buckle cover 710 and the bracket 708, and a gap between the second sealing ring 834 and the bracket 708 is installed to provide waterproof ability.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. An apparatus for cleaning skin of a user, the apparatus comprising:
a housing;
a brush assembly in the housing for cleaning the skin of the user, wherein the brush assembly is rotably installed in the housing;
a driving assembly to drive the brush assembly to rotate, the driving assembly is connected to the brush assembly; and
at least one stimulation element in the housing to provide a therapy to the user.

2. The apparatus of claim 1, wherein the brush assembly comprises a first brush disc and a second brush disc, the second brush disc is rotably sleeved outside the first brush disc, and the driving assembly drives the first brush disc to rotate differentially with the second brush disc.

3. The apparatus of claim 2, wherein the first brush disc comprises a support plate and a plurality of group of first bristles arranged on the support plate; and the second brush disc comprises a support ring and a plurality of group of second bristles are arranged on the support ring.

4. The apparatus of claim 3, wherein the plurality of group of first bristles and the plurality of group of second bristles are transparent or translucent.

5. The apparatus of claim 1 further comprising an annular seat, a first end of the annular seat is detachably connected with a liquid outlet of a bottle body and a second end of the annular seat is detachably connected with the housing.

6. The apparatus of claim 1, wherein the at least one stimulation element is arranged around axis of the brush assembly.

7. The apparatus of claim 1, wherein the at least one stimulation element is selected from a group consisting of Light Emitting Diodes (LEDs), lasers, heating elements, cooling elements, vibration elements, electrodes, micro-current element and combinations thereof.

8. An apparatus for cleaning skin of a user, the apparatus comprising:
a housing;
a brush assembly in the housing, the brush assembly comprising a first brush disc and a second brush disc, the second brush disc is roatably sleeved outside the first brush disc; and
a driving assembly connected with the brush assembly for driving the first brush disc to rotate differentially with the second brush disc.

9. The apparatus of claim 8, wherein the first brush disc comprises a support plate and a plurality of groups of first bristles arranged on the support plate; and the second brush disc comprises a support ring and the a plurality of group of second bristles are arranged on the support ring.

10. The apparatus of claim 9, wherein stiffness of the plurality of group of first bristles and the plurality of group of second bristles are different.

11. The apparatus of claim 8, wherein the driving assembly further comprises: a gear assembly for driving the first brush disc to synchronously reverse rotated with the second brush disc; and a driver for driving the gear assembly and a mounting pad for supporting the driver.

12. The apparatus of claim 11, wherein the gear assembly comprises the driver for driving the first brush disc to rotate, an internal tooth disc for driving the second brush disc to rotate and a transmission gear that connects the internal tooth disc with the gear assembly.

13. An apparatus for cleaning skin of a user, the apparatus comprising:
a housing;
a brush assembly in the housing for cleaning the skin of the user;
a driving assembly connected with the brush assembly to drive the brush assembly to rotate; and
a lifting assembly for driving the brush assembly to retract and stretch out from the housing.

14. The apparatus of claim 13, wherein the lifting assembly comprises a rotating seat and a lifting seat connected with the driving assembly, a first end of the rotating seat is rotably installed at top of the housing, and a second end of the rotating seat extends inside the housing and connects with the lifting seat.

15. The apparatus of claim 14, wherein the housing comprises a guide rail for guiding the lifting seat to move vertically towards and forwards the rotating seat, when the rotating seat is rotated.

16. The apparatus of claim 15, wherein the lifting seat comprise one or more connecting arm, the one or more arm connects with the guide rail.

17. The apparatus of claim 14, wherein the lifting assembly further comprises a spring for driving the lifting seat close to the rotating seat, the spring is connected with the lifting seat and the rotating seat.

18. The apparatus of claim 14, wherein the lifting assembly further comprises one or more slide rods connected to one or more connecting blocks in the lifting seat, the one or more slide rods extend vertically upward in the housing and connects to the rotating seat.

19. The apparatus of claim 18, wherein a first end of the one or more slide rods comprises a limiting part, the one or more slide rods has a spring sleeved.

20. The apparatus of claim 13 further comprises an annular seat, a first end of the annular seat is detachably connected with a liquid outlet end of a bottle body and a second end of the annular seat is detachably connected with the housing.
